(19) United States
(12) Patent Application Publication
(10) Pub. No.: US 2026/0284015 A1
(43) Pub. Date: Sep. 24, 2026

(54) DOSAGE REGIME OF CRINECERFONT FOR TREATING CONGENITAL ADRENAL HYPERPLASIA

(71) Applicant: Neurocrine Biosciences, Inc., San Diego, CA (US)

(72) Inventors: Jean L. Chan, San Diego, CA (US); Robert H. Farber, San Diego, CA (US)

(21) Appl. No.: 19/469,981

(22) PCT Filed: Mar. 29, 2024
(86) PCT No.: PCT/US2024/022158

§ 371 Date: Sep. 26, 2025

Related U.S. Application Data
(60) Provisional application No. 63/455,752, filed on Mar. 30, 2023.

(51) Int. Cl. A61K 31/426; A61K 31/573; A61P 5/38

(52) U.S. Cl. CPC A61K 31/426; A61K 31/573; A61P 5/38

(57) ABSTRACT

Provided are methods of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, in an adult subject having congenital adrenal hyperplasia.

### CROSS-REFERENCE TO RELATED APPLICATIONS

The present application claims the benefit of U.S. Provisional Application No. 63/455,752, filed Mar. 30, 2023, which is incorporated herein by reference in its entirety.

### FIELD OF THE INVENTION

Congenital adrenal hyperplasia (CAH) is a disease that includes a group of autosomal recessive disorders that result in an enzyme deficiency that alters the production of adrenal steroids due to 21-hydroxylase deficiency, a condition that results in little or no cortisol biosynthesis. One clinical manifestation of the absence of cortisol is the lack of feedback inhibition of pituitary adrenocorticotropic hormone (ACTH) secretion. Increased ACTH levels cause adrenal hyperplasia and the enzyme mutation causes a shunting of cortisol precursor steroids to alternate pathways. Most notably, the shunting of androgens leads to virilization and other developmental complications in females and the over-accumulation of ACTH is associated with the formation of testicular adrenal rest tumors in males. In addition, since the same enzyme (21-hydroxylase) is used in the pathway for the biosynthesis of the mineralocorticoids, a number of these patients suffer from aldosterone deficiency which can result in dehydration and death due to salt-wasting. The prevalence of classic 21-hydroxylase deficiency CAH in the US general population, based on newborn screening, has been documented as 1:10,000 to 1:20,800.

Currently, exogenous corticosteroids are the standard of care for treating patients with classic CAH. This treatment is used to correct the cortisol deficiency and reduce the excessive ACTH levels and androgen excess. However, the dose and duration of steroid use required to suppress ACTH are typically well above the normal physiological level used for cortisol replacement alone (as in patients with Addison's disease). This increased exposure to glucocorticoids can lead to iatrogenic Cushing's syndrome, increased cardiovascular risk factors, glucose intolerance, reduced growth velocity, and decreased bone mineral density in CAH patients.

It has been demonstrated in clinical trials that orally active compounds that block CRF₁, such as 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine (Compound A), provide a reduction from baseline in 17-hydroxyprogesterone (17-OHP) and androstenedione levels in amounts believed to allow use of lower, more physiologic doses of glucocorticoid (e.g. hydrocortisone) in patients with CAH. The structure of Compound A is shown below:

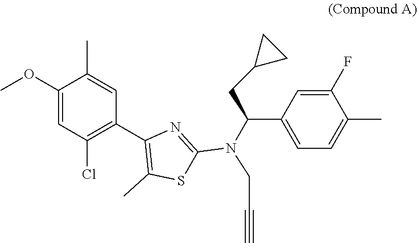

There is a significant, unmet need for methods for treating CAH. The present disclosure fulfills these and other needs, as evident in reference to the following disclosure.

### SUMMARY

Provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

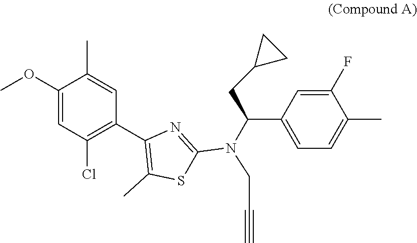

as an adjunct to glucocorticoid therapy in an adult subject having congenital adrenal hyperplasia (CAH), the method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is equivalent to about 200 mg of Compound A free base q.P.M.

Also provided is a method of treating congenital adrenal hyperplasia (CAH) in an adult subject already being treated with glucocorticoid therapy, the method comprising: administering to the subject a first amount of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

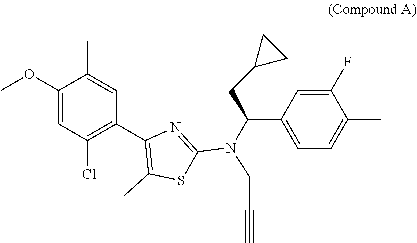

for a first period of time, and subsequently administering to the subject a second amount of Compound A, or a pharmaceutically acceptable salt thereof, for a second period of time, wherein the second amount is equivalent to about 200 mg of Compound A free base q.P.M.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure, or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

As used herein, in some embodiments, “pharmaceutically acceptable salt” refers to acid addition salts with an inorganic or an organic acid. Lists of suitable salts are found in WO 87/05297, Johnston et al., published Sep. 11, 1987; Remington's Pharmaceutical Sciences, 17th ed., Mack Publishing Company, Easton, Pa., 1985, p. 1418; and J. Pharm. Sci., 66, 2 (1977), each of which is incorporated herein by reference in its entirety. A reference for the preparation and selection of pharmaceutical salts of the present disclosure is P. H. Stahl & C. G. Wermuth “Handbook of Pharmaceutical Salts,” Verlag Helvetica Chimica Acta, Zurich, 2002, which is incorporated herein by reference in its entirety. The organic or inorganic acids include, but are not limited to, hydrochloric, hydrobromic, sulfuric, nitric, phosphoric, sulfamic, acetic, trifluoroacetic, trichloroacetic, propionic, hexanoic, cyclopentylpropionic, glycolic, glutaric, pyruvic, lactic, malonic, succinic, sorbic, ascorbic, malic, maleic, fumaric, tartaric, citric, benzoic, 3-(4-hydroxybenzoyl)benzoic, picric, cinnamic, mandelic, phthalic, lauric, methanesulfonic, ethanesulfonic, 1,2-ethane-disulfonic, 2-hydroxyethanesulfonic, benzenesulfonic, 4-chlorobenzenesulfonic, 2-naphthalenesulfonic, 4-toluenesulfonic, camphoric, camphorsulfonic, 4-methylbicyclo[2.2.2]-oct-2-ene-1-carboxylic, glucoheptonic, 3-phenylpropionic, trimethylacetic, tert-butylacetic, lauryl sulfuric, gluconic, benzoic, glutamic, hydroxynaphthoic, salicylic, stearic, cyclohexylsulfamic, quinic, muconic acid, and the like. In some embodiments, “pharmaceutically acceptable salt” refers to base addition salts with an inorganic or an organic base. Inorganic bases which may be used to prepare salts include, but are not limited to, for example, sodium, potassium, lithium, ammonium, calcium, magnesium, iron, zinc, manganese, aluminum hydroxides, carbonates, bicarbonates, phosphates, and the like. Organic bases from which may be used to prepare salts include, but are not limited to, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine.

As used herein, “about” means ±20% of the stated value, and includes more specifically values of ±10%, ±5%, ±2%, and ±1% of the stated value.

As used herein, “baseline” refers to the period of time just prior to initiation of therapy. The patient's condition just prior to initiation of therapy can be referred to as the patient's baseline condition.

As used herein, “adjusting administration”, “altering administration”, “adjusting dosing”, or “altering dosing” are all equivalent and mean tapering off, reducing or increasing the dose of the substance, ceasing to administer the substance to the patient, or substituting a different active agent for the substance.

As used herein, “administering to a patient” refers to the process of introducing a composition or dosage form into the patient via an art-recognized means of introduction.

As used herein, “adult” refers to a subject at least 18 years of age.

As used herein, “b.i.d.” refers to twice daily (doses separated by approximately 12 hours).

As used herein, “co-administer” and “co-administration” and variants thereof mean the administration of at least two drugs to a patient either subsequently, simultaneously, or consequently proximate in time to one another (e.g., within the same day, or week or period of 30 days, or sufficiently proximate that each of the at least two drugs can be simultaneously detected in the blood plasma). When co-administered, two or more active agents can be co-formulated as part of the same composition or administered as separate formulations. This also may be referred to herein as “concomitant” administration or variants thereof.

As used here, “congenital adrenal hyperplasia” or “CAH” refers to a group of genetic disorders that affect the adrenal glands. Congenital adrenal hyperplasia (CAH) is a group of autosomal recessive genetic disorders that result in little or no cortisol biosynthesis. One clinical manifestation of the absence of cortisol is the lack of feedback inhibition of CRF which causes dysregulation of the HPA axis. The most frequent form of the disease is 21-hydroxylase deficiency caused by mutations in the CYP21A2 gene located on chromosome 6p21, which accounts for approximately 95% of CAH cases, the 21-hydroxylase enzyme deficiency also causes a shunting of cortisol precursor steroids leading to excess androgen (e.g., 17-hydroxyprogesterone, androstenedione, and testosterone) product. These mutations can range from complete loss of enzyme activity required for synthesis of cortisol in the adrenal cortex to a spectrum of partial loss, which results in disease severity that is a direct consequence of a specific mutation. This continuum of 21-hydroxylase deficiency has been broadly classified into salt-wasting and simple-virilizing forms, grouped as classic (or classical) CAH, and the milder form known as non-classic (or non-classical) CAH (NCCAH) or “late-onset” CAH, which is usually diagnosed in late childhood or early adulthood. Non-classic CAH patients are either homozygous or compound heterozygotes, often with a classical CAH allele. These patients have sufficient enzyme activity (>20-50% of normal) such that they do not have salt-wasting or cortisol deficiency and have normal genitalia at birth, and many remain asymptomatic throughout life. In the less frequent form of the disease, which accounts for 5% of cases, mutation of the 11β-hydroxylase gene CYP11B1 results in CAH (11β-OH CAH). Classic CAH is a rare disease; the incidence of classic CAH is estimated to be ~1:15,000 worldwide. Approximately 75% of patients with classic CAH have the salt wasting form of the disease, characterized by severe enzyme deficiency and insufficient levels of cortisol and aldosterone. Approximately 25% of patients with classic CAH have the simple virilizing form, in which cortisol synthesis is impaired, but enzyme activity is sufficient for adequate production of aldosterone.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not Compound A free base, the quantity is the molar equivalent to the corresponding amount of Compound A free base. For example, often a drug is packaged in a pharmaceutically acceptable salt form, and the dosage for strength refers to the mass of the molar equivalent of the corresponding free base, Compound A.

As used herein, “dosing regimen” means the dose of an active agent taken at a first time by a patient and the interval (time or symptomatic) at which any subsequent doses of the active agent are taken by the patient such as from about 100 mg to about 300 mg once daily or from about 20 mg to about 160 mg twice daily, e.g., about 100 mg, about 150 mg, about 200 mg, about 250 mg, or about 300 mg once daily; or about 20 mg, about 40 mg, about 60 mg, about 80 mg, about 100 mg, about 120 mg, or about 160 mg twice daily. The additional doses of the active agent can be different from the dose taken at the first time. For example, a first dose may range from about 20 mg to about 160 mg, e.g., about 20 mg, about 40 mg, about 60 mg, about 80 mg, about 100 mg, about 120 mg, or about 160 mg; and a second dose may range from about 100 mg to about 300 mg, e.g., about 100 mg, about 150 mg, about 200 mg, about 250 mg, or about 300 mg.

As used herein, a “dosage” is the prescribed administration of a specific amount, number, and frequency of doses over a specific period of time.

As used herein, “effective amount” and “therapeutically effective amount” of an agent, compound, drug, composition, or combination is an amount which is nontoxic and effective for producing some desired therapeutic effect upon administration to a subject or patient (e.g., a human subject or patient). The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, “informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

As used herein, “labeling” means all labels or other means of written, printed, graphic, electronic, verbal, or demonstrative communication that is upon a pharmaceutical product or a dosage form or accompanying such pharmaceutical product or dosage form.

As used herein, “a medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, “Medication Guide” means an FDA-approved patient labeling for a pharmaceutical product conforming to the specifications as set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

As used herein, “patient” or “individual” or “subject” means a mammal, including a human, for whom or which therapy is desired, and generally refers to the recipient of the therapy.

As used herein, “patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

As used herein, “pharmaceutically acceptable” refers to a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” include acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

As used herein, a “product” or “pharmaceutical product” means a dosage form of an active agent plus published material, and optionally packaging.

As used herein, “product insert” means the professional labeling (prescribing information) for a pharmaceutical product, a patient package insert for the pharmaceutical product, or a medication guide for the pharmaceutical product.

As used herein, “professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example, indication and usage; dosage and administration; who should take it; adverse events (side effects); instructions for use in special populations (pregnant women, children, geriatric, etc.); safety information for the patient, and the like.

As used herein, “published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “q.A.M.” means every day in the morning.

As used herein, “q.P.M.” means every day in the evening.

As used herein, “risk” means the probability or chance of adverse reaction, injury, or other undesirable outcome arising from a medical treatment. An “acceptable risk” means a measure of the risk of harm, injury, or disease arising from a medical treatment that will be tolerated by an individual or group. Whether a risk is “acceptable” will depend upon the advantages that the individual or group perceives to be obtainable in return for taking the risk, whether they accept whatever scientific and other advice is offered about the magnitude of the risk, and numerous other factors, both political and social. An “acceptable risk” of an adverse reaction means that an individual or a group in society is willing to take or be subjected to the risk that the adverse reaction might occur since the adverse reaction is one whose probability of occurrence is small, or whose consequences are so slight, or the benefits (perceived or real) of the active agent are so great. An “unacceptable risk” of an adverse reaction means that an individual or a group in society is unwilling to take or be subjected to the risk that the adverse reaction might occur upon weighing the probability of occurrence of the adverse reaction, the consequences of the adverse reaction, and the benefits (perceived or real) of the active agent. “At risk” means in a state or condition marked by a high level of risk or susceptibility. Risk assessment consists of identifying and characterizing the nature, frequency, and severity of the risks associated with the use of a product.

As used herein, “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

Provided is a method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

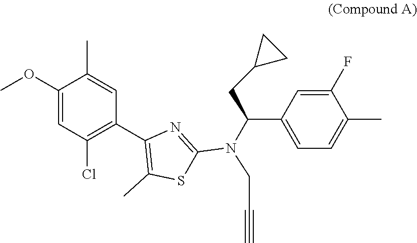

as an adjunct to glucocorticoid therapy in an adult subject having congenital adrenal hyperplasia (CAH), the method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is equivalent to about 200 mg of Compound A free base q.P.M.

Also provided is a method of treating congenital adrenal hyperplasia (CAH) in an adult subject already being treated with glucocorticoid therapy, the method comprising: administering to the subject a first amount of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof,

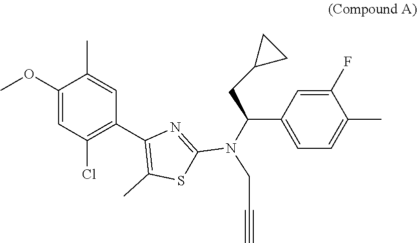

for a first period of time, and subsequently administering to the subject a second amount of Compound A, or a pharmaceutically acceptable salt thereof, for a second period of time, wherein the second amount is equivalent to about 200 mg of Compound A free base q.P.M.

In some embodiments, administering Compound A, or a pharmaceutically acceptable salt thereof, refers to administration of Compound A, or a pharmaceutically acceptable salt thereof, with the subject's breakfast and evening meal.

In some embodiments, the first amount is equivalent to about 100 mg of Compound A free base b.i.d. In some embodiments, the first amount is equivalent to about 100 mg of Compound A free base q.A.M. and to about 100 mg of Compound A free base q.P.M.

In some embodiments, the first amount is equivalent to about 100 mg of Compound A free base q.A.M. and to about 200 mg of Compound A free base q.P.M.

In some embodiments, the first period of time is between 1 week and 1 year. In some embodiments, the first period of time is at least 6 months. In some embodiments, the first period of time is less than 6 months. In some embodiments, the first period of time is 24 hours. In some embodiments, the first period of time is less than 24 hours. In some embodiments, the first period of time is more than 6 months. In some embodiments, the first period of time is about 6 months. In some embodiments, the first period of time is at least one year. In some embodiments, the first period of time is greater than one year. In some embodiments, the first period of time is about one year.

In some embodiments, the second amount is administered because of a missed dose in the first amount. In some embodiments, the missed dose in the first amount is the q.A.M. dose. In some embodiments, the second amount is administered on the same day as the dose was missed in the first amount. In some embodiments, the missed dose in the first amount is the q.P.M. dose. In some embodiments, the second amount is administered on the next day as the dose was missed in the first amount. In some embodiments, the dose of the second amount is adjusted to the first amount after the second period of time. In some embodiments, the dose of the second amount is adjusted to the first amount after the second period of time because of a missed dose in the first amount. In some embodiments, the second period of time is less than 24 hours. In some embodiments, the second period of time is about 12 hours. In some embodiments, the q.A.M. dose of the first amount is missed. In some embodiments, in the event of a missed first amount q.A.M. dose, the second amount is administered in the evening, and then the regular first amount dosing schedule is resumed.

In some embodiments, the second period of time is between 1 week and 1 year. In some embodiments, the second period of time is at least 6 months. In some embodiments, the second period of time is less than 6 months. In some embodiments, the second period of time is 24 hours. In some embodiments, the second period of time is less than 24 hours. In some embodiments, the second period of time is more than 6 months. In some embodiments, the second period of time is about 6 months. In some embodiments, the second period of time is at least one year. In some embodiments, the second period of time is greater than one year. In some embodiments, the second period of time is about one year.

In some embodiments, the method further comprises adjusting for a missed dose in the first amount. In some embodiments, the missed first amount dose is the q.A.M. dose. In some embodiments, the missed first amount dose is the q.P.M. dose. In some embodiments, the method further comprises adjusting for a missed dose in the second amount. In some embodiments, in the event of a missed dose, the amount of the missed dose is administered as soon as possible, even if it is soon before the next scheduled dose, and then the regular dosing schedule is resumed.

In some embodiments, the glucocorticoid therapy is a daily dose of a glucocorticoid administered to the subject.

In some embodiments, the daily dose of the glucocorticoid is administered once daily or through multiple administrations. In some embodiments, the daily dose of the glucocorticoid is administered once daily. In some embodiments, the daily dose of the glucocorticoid is administered through multiple administrations.

In some embodiments, the glucocorticoid is selected from hydrocortisone, prednisone, methylprednisolone, prednisolone, and dexamethasone, or a combination thereof.

In some embodiments, prior to the administering of the amount equivalent to about 200 mg of Compound A free base q.P.M., the subject was receiving a glucocorticoid at a target dose.

In some embodiments, the target dose of glucocorticoid treatment is ≤11 mg/m²/day hydrocortisone equivalents.

In some embodiments, the target dose of glucocorticoid treatment is about 8 mg/m²/day to about 10 mg/m²/day hydrocortisone equivalents.

In some embodiments, the target dose of glucocorticoid treatment is about 8 mg/m²/day hydrocortisone equivalents.

In some embodiments, the glucocorticoid is hydrocortisone. In some embodiments, the glucocorticoid is methylprednisolone using a hydrocortisone dose equivalent conversion factor of 4×. In some embodiments, the glucocorticoid is prednisolone using a hydrocortisone dose equivalent conversion factor of 4×. In some embodiments, the glucocorticoid is prednisone using a hydrocortisone dose equivalent conversion factor of 4×. In some embodiments, the glucocorticoid is dexamethasone using a hydrocortisone dose equivalent conversion factor of 60×.

In some embodiments, prior to the administering of the amount equivalent to about 200 mg of Compound A free base q.P.M., the subject demonstrated adequate disease control.

In some embodiments, prior to the administering of the amount equivalent to about 200 mg of Compound A free base q.P.M., the subject demonstrated adequate disease control and was being administered a glucocorticoid at the target dose.

In some embodiments, the subject demonstrated adequate disease control if their androstenedione levels are ≤120% of the subject's baseline or ≤upper limit of normal (ULN) for age and sex of the subject.

In some embodiments, the subject demonstrated adequate disease control in the opinion of the investigator.

In some embodiments, the method further comprises monitoring the subject for adequate disease control, wherein if the disease control is inadequate and the glucocorticoid therapy is at or above the target dose of the subject, then the amount of Compound A, or a pharmaceutically acceptable salt thereof, is adjusted to an amount equivalent to about 100 mg of Compound A free base b.i.d.

In some embodiments, the method further comprises monitoring the subject for adequate disease control, wherein if the disease control is inadequate and the glucocorticoid treatment is at or above the target dose of the subject, then the amount of Compound A, or a pharmaceutically acceptable salt thereof, is adjusted to an amount equivalent to about 100 mg of Compound A free base q.A.M. and an amount equivalent to about 200 mg of Compound A free base q.P.M.

In some embodiments, the subject demonstrated inadequate disease control if their androstenedione levels are >120% of the subject's baseline or >upper limit of normal (ULN) for age and sex of the subject.

In some embodiments, the subject demonstrated inadequate disease control in the opinion of the investigator.

In some embodiments, the target dose of glucocorticoid treatment is ≤11 mg/m²/day hydrocortisone equivalents.

In some embodiments, the target dose of glucocorticoid treatment is about 8 mg/m²/day to about 10 mg/m²/day hydrocortisone equivalents.

In some embodiments, the target dose of glucocorticoid treatment is about 8 mg/m²/day hydrocortisone equivalents.

In some embodiments, the glucocorticoid is hydrocortisone. In some embodiments, the glucocorticoid is methylprednisolone using a hydrocortisone dose equivalent conversion factor of 4×. In some embodiments, the glucocorticoid is prednisolone using a hydrocortisone dose equivalent conversion factor of 4×. In some embodiments, the glucocorticoid is prednisone using a hydrocortisone dose equivalent conversion factor of 4×. In some embodiments, the glucocorticoid is dexamethasone using a hydrocortisone dose equivalent conversion factor of 60×.

In some embodiments, Compound A is a free base.

In some embodiments, Compound A is administered with food.

In some embodiments, Compound A is administered to the subject in a fed state.

In some embodiments, the CAH is classical Congenital Adrenal Hyperplasia.

In some embodiments, the CAH is non-classical Congenital Adrenal Hyperplasia.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1

A randomized, double-blind, placebo-controlled study to evaluate the safety and efficacy of Compound A in adult subjects with classic congenital adrenal hyperplasia, followed by open-label treatment will be conducted. Specifically, this is a Phase 3, randomized, double-blind, placebo-controlled study to evaluate the efficacy, safety, and tolerability of Compound A versus placebo administered b.i.d. with breakfast and the evening meal (doses separated by approximately 12 hours) for 24 weeks in approximately 165 adult subjects with classic CAH due to 21-hydroxylase deficiency. Eligible subjects will be randomly assigned in a 2:1 ratio (active:placebo) to 2 treatment groups, Compound A 100 mg b.i.d. or placebo. After the 24-week randomized treatment period, there will be a 6 month, open-label treatment period, during which all subjects will receive Compound A 100 mg b.i.d. At Month 12, subjects who have not reduced their glucocorticoid dose to ≤11 mg/m²/day will be re-randomized (2:1) to receive 100 mg every morning (q.A.M.) and 200 mg every evening (q.P.M.) or to continue 100 mg b.i.d., in a blinded fashion. Subjects who have reduced their glucocorticoid dose to ≤11 mg/m²/day will continue to receive 100 mg b.i.d. in an open label fashion. At Month 18, subjects will review applicable portions of the informed consent form and confirm whether they will participate in the optional open-label extension (OLE) treatment period for continued access to Compound A.

During the OLE, subjects will have their glucocorticoid doses adjusted as appropriate and tolerated to achieve the lowest glucocorticoid dose that maintains adequate disease control (in the opinion of the investigator). The glucocorticoid dose reduction will not require dose reduction below 8 mg/m²/day hydrocortisone equivalents.

Starting at Month 18, all subjects who are continuing in the OLE will initially receive Compound A 100 mg b.i.d. If the subject has inadequate disease control despite receiving glucocorticoid treatment at their target dose (in the opinion of the investigator), the Compound A dose may be increased to 100 mg q.A.M. and 200 mg q.P.M. After the Month 24 visit, an alternative dosing regimen of once daily 200 mg q.P.M. can be considered per the investigator.

### Screening Period (Weeks −4 Up to Day −1)

Subjects will undergo screening for up to 4 weeks (Weeks −4 to Day −1) to determine eligibility, with assessments performed according to the Schedule of Assessments. There will be a second visit (at home or the study site) during the screening period to collect a blood sample (for hormone measurements). The screening period may be extended by 2 weeks (if needed) for results of Screening Visit 2 hormone tests. Subjects must be on a supraphysiologic glucocorticoid regimen defined as >13 mg/m²/day in hydrocortisone dose equivalents (Table 1) adjusted for body surface area (BSA) that has been stable at least 1 month leading up to screening.

TABLE 1
Hydrocortisone Dose Equivalent Conversion Table

| | Hydrocortisone dose |
|---|---|
| Glucocorticoid | equivalent conversion |
| Methylprednisolone | 4× |
| Prednisolone | 4× |
| Prednisone | 4× |
| Dexamethasone | 60× |

See, Auchus and Arlt, J Clin Endocrinol Metab. 2013 July; 98(7): 2645-55; Speiser et al., J Clin Endocrinol Metab. 2018 Nov. 1; 103(11): 4043-88.

The glucocorticoid regimen should be optimized by the treating physician to achieve control of adrenal androgen levels and minimization of glucocorticoid dosage to the extent appropriate for the subject's individual medical needs and treatment goals.

Rescreening is permitted if a subject does not meet all eligibility requirements and returns to be rescreened. A subject that has failed screening twice may not be rescreened again without prior permission from the Medical Monitor.

### Randomized, Double-Blind, Placebo-Controlled Treatment Period (Day 1 Up to Week 24)

### 4-Week Glucocorticoid Stable Period (Day 1 Up to Week 4)

During the first 4 weeks of the study, subjects should maintain their stable glucocorticoid regimen, except for sick-day guidelines (see, e.g., El-Maouche D et al., J Clin Endocrinol Metab. 2018 Jun. 1; 103 (6): 2336-2345).

On Day 1 (baseline), subjects will collect a urine sample (all voids from midnight the night before the study visit to the first morning void after awakening for the day) at home in the morning and bring it to the site for measurement of androgen metabolite levels. They will hold their morning glucocorticoid dose and bring it with them to the study site so that a blood sample can be obtained prior to taking the morning glucocorticoid dose; subjects will then take their morning dose of glucocorticoid at the study site, and another blood sample will be taken approximately 2 hours postdose in order to establish the baseline pre- and post-glucocorticoid hormone levels. Subjects should be fasting from the night before so that fasting blood tests and an oral glucose tolerance test can be performed, but should be encouraged to drink water to avoid any hypovolemic status.

Subjects will be randomized on Day 1 in a 2:1 ratio (active:placebo). Randomization will be stratified by total daily glucocorticoid dose, glucocorticoid type, and sex. Beginning on Day 1 (baseline), study drug will be administered at home with the subject's evening meal; thereafter, study drug will be administered b.i.d. with the subject's breakfast and evening meal (doses separated by approximately 12 hours).

### 8-Week Glucocorticoid Reduction Period (Week 4 Up to Week 12)

During this period, subjects will undergo a down-titration (in 4 or fewer steps) of their glucocorticoid dose with the goal to reach a target dose of 8 mg/m²/day to 10 mg/m²/day (hydrocortisone equivalents adjusted for BSA) by Week 12, unless the subject has any signs or symptoms suggestive of clinically relevant glucocorticoid insufficiency or unacceptable symptoms of hyperandrogenism (see below for additional details on the glucocorticoid dose reduction schedule).

At the Week 4 visit, a similar procedure will be followed as for Day 1 to obtain a more detailed assessment of androgen status, with collection of a urine sample at home and collection of blood samples prior to and approximately 2 hours after dosing of morning glucocorticoid and study drug at the study site. At this visit, the investigator will instruct the subject on the first step of the glucocorticoid dose reduction and arrange to contact the subject by telephone within a week of the study visit to assess how the subject is tolerating the glucocorticoid dose reduction. During the follow-up telephone contact, if the investigator feels that a clinical assessment and/or laboratory tests are needed, these can be performed as an unscheduled visit.

Subjects will have study visits at Weeks 6 (at home or the study site), 9 (at home or the study site), and 12 for study assessments, including collection of blood samples to assess hormone levels and routine safety assessments.

At the Week 6 visit, the investigator will instruct the subject on the second step of the glucocorticoid dose reduction (if applicable) and, if a glucocorticoid dose reduction occurred, will arrange to contact the subject by telephone within a week of the study visit to assess how the subject is tolerating the glucocorticoid dose reduction. The investigator will contact the subject at approximately Week 8 to advise on the third step of glucocorticoid dose reduction (if applicable).

At the Week 9 study visit, the investigator will assess whether the subject is tolerating the third glucocorticoid dose reduction (if applicable). The investigator will contact the subject at approximately Week 10 to advise on the fourth step of glucocorticoid dose reduction (if applicable) and, if a glucocorticoid dose reduction occurred, will arrange to contact the subject by telephone within a week of the study visit to assess how the subject is tolerating the glucocorticoid dose reduction.

If the subject experiences any of the following signs or symptoms at any time during the glucocorticoid dose reduction process, the glucocorticoid dose should NOT be reduced further but returned to the previous dose that was tolerated. However, before the glucocorticoid dose reduction is stopped for symptoms or signs of orthostatic hypotension, volume status should be optimized (e.g., with additional dietary salt, salt tablets, intravenous saline).
Unexplained hyponatremia (serum sodium <135 mmol/L)
Orthostatic hypotension with decrease in systolic blood pressure >20 mmHg or in diastolic blood pressure >10 mmHg after standing (from a seated position) after approximately 2 minutes, or severe symptoms of dizziness or lightheadedness upon standing
Severe nausea, food aversion, vomiting
Unacceptable symptoms of hyperandrogenism (e.g., hirsutism, acne, amenorrhea)

Glucocorticoid dose reductions during Weeks 4 to 12 should proceed even if androstenedione levels increase transiently, provided that the increase is asymptomatic and tolerated by the subject.

At the Week 12 visit, based on review of the subject's hormone levels collected up to that visit as well as based on clinical assessment, the investigator will determine the appropriate dose of glucocorticoid to continue past Week 12 (the reduced dose if tolerated, or a prior [higher] dose) in order to achieve adequate control of androgen levels (i.e., androstenedione ≤120% of the subject's baseline or ≤upper limit of normal [ULN] for age and sex).

### 12-Week Glucocorticoid Optimization Period (Week 12 Up to Week 24)

Subjects will continue on the glucocorticoid regimen as instructed by the investigator at Week 12 and return to the study site at Week 16 (at home or the study site), Week 20 (at home or the study site), and Week 24 during the glucocorticoid optimization period. At these visits, the investigator will review the laboratory results from the preceding study visit and determine if the glucocorticoid regimen requires adjustment in order to achieve adequate control of androgen levels (i.e., androstenedione ≤120% of the subject's baseline or ≤ULN for age and sex).

At the Week 24 visit, subjects will follow a similar procedure as Day 1 for additional androgen assessments with collection of a urine sample at home and collection of blood samples prior to and approximately 2 hours after dosing of morning glucocorticoid and study drug at the study site. Subjects should be fasting from the night before, but should be encouraged to drink water to avoid any hypovolemic status, and a glucose tolerance test will be performed (with study drug taken with the glucose load rather than a meal).

### Open-Label Treatment Period (Week 24 Up to Month 12)

For the purpose of this study, months are defined as 4-week intervals.

Starting the evening of the Week 24 visit (after all Week 24 assessments have been performed), all subjects will receive active study drug (Compound A) 100 mg b.i.d. with breakfast and evening meals. Subjects should continue the glucocorticoid regimen specified by the investigator at Week 24. Subjects and investigators will remain blinded to subjects' treatment group assignment from the double-blind period.

### 1-Month Glucocorticoid Stable Period (Week 24 up to Month 7)

During the first month of open-label treatment with Compound A, subjects should maintain a stable glucocorticoid regimen (except for sick-day guidelines).

### 3-Month Glucocorticoid Reduction Period (Month 7 up to Month 10)

At Months 7 (at home or the study site), 8, and 9 (at home or the study site), investigators will decrease glucocorticoid doses in those subjects whose glucocorticoid dose is still greater than 11 mg/m²/day at Month 7 (unless there is a safety concern with regard to glucocorticoid insufficiency), with the goal to achieve a target physiologic dose of 8 to 10 mg/m²/day by Month 10. The glucocorticoid dose should be reduced by approximately 10% to 20% at each visit (Months 7, 8, and 9), as long as androstenedione levels are controlled (i.e., androstenedione ≤120% of the subject's baseline or ≤ULN for age and sex) and the subject is not experiencing any signs or symptoms suggestive of clinically relevant glucocorticoid insufficiency or unacceptable symptoms of hyperandrogenism. The glucocorticoid dose reduction will not require dose reduction below 8 mg/m²/day hydrocortisone equivalents. After each of the glucocorticoid dose reduction steps, the site should contact the subject by telephone (within a week) to assess how the subject is tolerating the glucocorticoid dose reduction. Subjects will have study visits at Months 8, 9, and 10 for study assessments including collection of blood samples for hormone levels.

### 2-Month Glucocorticoid Maintenance Period (Month 10 up to Month 12)

Subjects will have study assessments at Month 10 (at home or the study site) and Month 12 as outlined in the Schedule of Assessments. During this period, the goal should be to maintain stable glucocorticoid doses; however, the dose can be adjusted according to standard of care (e.g., to achieve the control of androgen levels appropriate to the treatment targets for each subject).

At the Month 12 visit, subjects will have additional androgen assessments with collection of a urine sample at home and blood sample collection before and approximately 2 hours after dosing of morning glucocorticoid and study drug at the study site. Subjects should be fasting from the night before (subjects should be encouraged to drink water to avoid any hypovolemic status). A glucose tolerance test will be performed (with study drug taken with the glucose load rather than a meal) at the Month 12 visit.

### Open-Label or Double-Blind Active-Controlled Treatment (Month 12 to Month 18)

6-Month Glucocorticoid Maintenance Period (Month 12 to Month 18) for Subjects with Month 12 Glucocorticoid Dose ≤11 mg/m²/day

Subjects with glucocorticoid dose ≤11 mg/m²/day at Month 12 will continue on active study drug at 100 mg b.i.d. until Month 18 with study visits at Months 14 (at home or the study site), 16 (at home or the study site), and 18. The goal during this period is to maintain stable glucocorticoid doses while androstenedione levels are controlled (i.e., androstenedione ≤120% of the subject's baseline or ≤ULN for age and sex), although the dose can be adjusted according to standard of care.

At the Month 18 visit, subjects will have additional androgen assessments with collection of a urine sample at home and blood sample collection before and approximately 2 hours after dosing of morning glucocorticoid and study drug at the study site. Subjects should be fasting from the night before (subjects should be encouraged to drink water to avoid any hypovolemic status).

6-Month Glucocorticoid Reduction/Optimization (Month 12 to Month 18) for Subjects with Month 12 Glucocorticoid Dose >11 mg/m²/day

Subjects with glucocorticoid dose >11 mg/m²/day at Month 12 will be re-randomized (2:1) to adjust active study drug dose to 100 mg q.A.M. and 200 mg q.P.M. or to continue active study drug at 100 mg b.i.d.; study drug will be blinded such that all re-randomized subjects will be taking the same number of capsules. Subjects should maintain a stable glucocorticoid regimen (except for sick-day guidelines) for the first month until the Month 13 visit. At the Month 13 visit, subjects will have additional androgen assessments with collection of a urine sample at home and blood sample collection before and approximately 2 hours after dosing of morning glucocorticoid and study drug at the study site.

At Months 13, 14 (at home or the study site), and 16 (at home or the study site), investigators will decrease glucocorticoid doses with the goal to achieve a target physiologic dose of 8 to 10 mg/m²/day by Month 18. The glucocorticoid dose should be reduced by approximately 10% to 20% at Months 13, 14, and 16, as long as androstenedione levels are controlled (i.e., androstenedione ≤120% of the subject's baseline or ≤ULN for age and sex) and the subject is not experiencing any signs or symptoms suggestive of clinically relevant glucocorticoid insufficiency or unacceptable symptoms of hyperandrogenism. The glucocorticoid dose reduction will not require dose reduction below 8 mg/m²/day hydrocortisone equivalents. After each of the glucocorticoid dose reduction steps, the site should contact the subject by telephone (within a week) to assess how the subject is tolerating the glucocorticoid dose reduction.

At the Month 18 visit, subjects will have additional androgen assessments with collection of a urine sample at home and blood sample collection before and approximately 2 hours after dosing of morning glucocorticoid and study drug at the study site. Subjects should be fasting from the night before (subjects should be encouraged to drink water to avoid any hypovolemic status).

### Open-Label Extension (OLE) Treatment Period (Month 18 Onwards)

At Month 18, subjects will review applicable portions of the informed consent form and confirm whether they will participate in the optional OLE.

During the OLE, subjects will have their glucocorticoid doses adjusted as appropriate and tolerated to achieve the lowest glucocorticoid dose that maintains adequate disease control (in the opinion of the investigator). The glucocorticoid dose reduction will not require dose reduction below 8 mg/m²/day hydrocortisone equivalents. After each glucocorticoid dose reduction, the site should contact the subject by telephone (within a week) to assess how the subject is tolerating the glucocorticoid dose reduction.

Starting at Month 18, all subjects who are continuing in the OLE will initially receive open-label Compound A 100 mg b.i.d. If the subject has inadequate disease control despite receiving glucocorticoid treatment at their target dose (in the opinion of the investigator), the Compound A dose may be increased to 100 mg q.A.M. and 200 mg q.P.M. (including at Month 18, after laboratory results are available). If the increased dose of 100 mg q.A.M. and 200 mg q.P.M. is not well tolerated, the dose may be reduced back to 100 mg b.i.d. After the Month 24 visit, an alternative dosing regimen of once daily 200 mg q.P.M. can be considered per the investigator. Compound A doses should generally only be adjusted at or shortly after study visits (after laboratory results are available).

In the setting of inadequate disease control, if the glucocorticoid dose is at or above the target, an increase in the glucocorticoid dose should generally be considered only after the Compound A dose has been maximized for the subject. Changes to the glucocorticoid and Compound A doses should generally be separated by at least 1 month in order to assess the effect of each change.

Study visits during the OLE will occur every 3 months until Month 24, and every 6 months thereafter, with the option of having the Month 21 visit at home. At the Month 24 visit, and every 12 months thereafter, subjects will have blood sample collection before and approximately 2 hours after dosing of morning glucocorticoid and study drug at the study site. Subjects should be fasting from the night before (subjects should be encouraged to drink water to avoid any hypovolemic status). A glucose tolerance test will also be performed at Month 24 and every 12 months thereafter (with study drug taken with the glucose load rather than a meal).

Subjects will remain in the OLE until Compound A becomes commercially available, the Sponsor elects to discontinue development of Compound A for CAH, the Sponsor elects to discontinue the study, or the subject meets one of the study withdrawal criteria.

### Follow-Up Period

A final posttreatment visit will be conducted approximately 1 month after subject's final dose of study drug.

### Study Assessments and Study Visit Scheduling

Efficacy, safety, and PK will be assessed at scheduled times throughout the study. As much as possible, all study visits (including baseline and follow-up) should occur at approximately the same time in the morning to standardize time of day for assessment of efficacy, safety, and drug exposure.

In the double-blind, placebo-controlled portion of the study, all visits after Day 1 during the glucocorticoid stable period and glucocorticoid reduction period have a visit window of ±5 days, and all visits during the glucocorticoid optimization period have a visit window of ±5 days. In the open-label treatment period, visits from Month 7 to Month 10 have a visit window of ±5 days and visits from Month 12 to Month 18 will have a visit window of ±7 days. During the OLE, visits will have a visit window of ±14 days. If a subject's glucocorticoid regimen is adjusted due to sick-day guidelines, the subject should resume their glucocorticoid dosing regimen for at least 3 days before their next scheduled hormone panel assessment, and this 3-day window supersedes all other visit windows.

Objectives of the study may include:
To evaluate the efficacy of Compound A (100 mg twice daily [b.i.d.]), compared with placebo, in reducing daily glucocorticoid dosage while maintaining adrenal androgen control.
To evaluate the efficacy of Compound A, compared with placebo, in reducing adrenal steroid levels following an initial 4-week treatment period.
To evaluate the effect of Compound A, compared with placebo, on clinical endpoints associated with supraphysiologic glucocorticoid dosing.
To evaluate plasma concentrations of Compound A and metabolites.
To assess the safety and tolerability of Compound A.
To evaluate an alternate dosing regimen of Compound A in subjects who have not reduced their glucocorticoid dose by Month 12.

### Criteria for Evaluation:

Efficacy criteria that may be evaluated may include:
Daily glucocorticoid regimen expressed in hydrocortisone equivalents adjusted for body surface area (BSA) (mg/m²/day).
Hormone measurements: 17-hydroxyprogesterone (17-OHP) (serum; ng/dL), androstenedione (serum; ng/dL), testosterone (serum; ng/dL), adrenocorticotropic hormone (ACTH) (plasma; pg/mL), cortisol (serum; μg/dL), luteinizing hormone (LH) (serum; IU/L), follicle stimulating hormone (FSH) (serum; IU/L), progesterone (serum; ng/mL), plasma renin activity (measured upright) (ng/mL/hr).
Urine androgen metabolite levels (androsterone and etiocholanolone).
Metabolic assessments (fasting lipid panel, homeostatic model assessment of insulin resistance [HOMA-IR] based on fasting glucose and insulin levels, glycated hemoglobin [HbA1c], glucose tolerance test).
Dual-energy X-ray absorptiometry (DXA) scan (bone mineral density and body composition).
Blood pressure.
Hirsutism and Acne Scales (female subjects only).
Testicular ultrasounds (to detect adrenal rest tissue) (male subjects only).
Menstrual Cycle Questionnaire (only in female subjects of childbearing potential who are not on hormonal or intrauterine device contraceptives).
Bone markers: serum osteocalcin, serum bone-specific alkaline phosphatase, serum C-terminal telopeptide, urine N-terminal telopeptide.

Patient-Reported Outcomes that may be evaluated may include 36-Item Short Form Health Survey (SF-36), EQ-5D-5L, Multidimensional Assessment of Fatigue (MAF), Psychological General Well-Being Index (PGWBI), and Medical Outcomes Study 12-Item Sleep Scale (MOS-12).

Pharmacokinetic criteria that may be evaluated may include: blood samples to evaluate plasma concentrations of Compound A and metabolites will be collected throughout the study.

Safety and tolerability will be monitored throughout the study and may include the following assessments:
Adverse events (including glucocorticoid-related events)
Clinical laboratory tests
Vital signs
Weight/body mass index (BMI), and waist circumference
Physical examinations
12-lead electrocardiograms
Brief Psychiatric Rating Scale (BPRS)
Columbia-Suicide Severity Rating Scale (C-SSRS)

Table 2 summarizes the open-label extension (OLE) treatment period protocol.

TABLE 2
Open-Label Extension (OLE) Treatment Period Protocol

| | Current | Current | |
|---|---|---|---|
| Disease | Glucocorticoid | Compound | |
| Control^{a} | Dose | A Dose | Action |
| Adequate^{a} | At target^{a} | Any | Continue current |
| | | | glucocorticoid and |
| | | | Compound A doses; OR |
| | | | If taking Compound A |
| | | | 100 mg qAM AND |
| | | | 200 mg qPM, |
| | | | decrease Compound A |
| | | | dose to 100 mg bid; OR |
| | | | After Month 24, option |
| | | | to dose Compound A as |
| | | | 200 mg qPM |
| | Above target^{a} | Any | Decrease glucocorticoid |
| | | | dose towards target^{b} |
| Inadequate^{a} | At or above | 100 mg | Discuss compliance with |
| | target^{a} | bid | glucocorticoid and study |
| | | | drug dosing; OR |
| | | | Increase Compound A |
| | | | dose to 100 mg qAM |
| | | | and 200 mg qPM |
| | | 200 mg | Discuss compliance with |
| | | qPM | glucocorticoid and study |
| | | | drug dosing; OR |
| | | | Change Compound A |
| | | | dose to 100 mg bid; OR |
| | | | Increase Compound A |
| | | | dose to 100 mg qAM |
| | | | and 200 mg qPM |
| | | 100 mg | Discuss compliance with |
| | | qAM AND | glucocorticoid and study |
| | | 200 mg | drug dosing; OR |
| | | qPM^{c} | Increase glucocorticoid |
| | | | dose as clinically |
| | | | indicated |
| | Below target^{a} | Any | Increase glucocorticoid |
| | | | dose towards target |

^{a}In the opinion of the investigator.
^{b}Unless the investigator deems further attempts at dose reduction to not be advisable for that subject. After each glucocorticoid dose reduction, the site should contact the subject by telephone (within a week) to assess how the subject is tolerating the glucocorticoid dose reduction.
^{c}Or the maximum Compound A dose tolerated by the subject.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, or a pharmaceutically acceptable salt thereof, 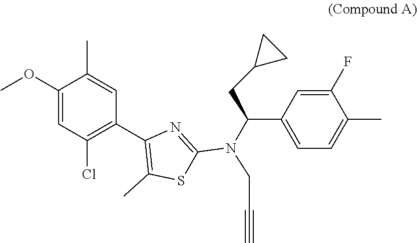
as an adjunct to glucocorticoid therapy in an adult subject having congenital adrenal hyperplasia (CAH), said method comprising: administering to the subject a first amount of Compound A, or a pharmaceutically acceptable salt thereof, for a first period of time, and subsequently administering to the subject a second amount of Compound A, or a pharmaceutically acceptable salt thereof, for a second period of time, wherein the second amount is equivalent to about 200 mg of Compound A free base q.P.M, and wherein the first amount of Compound A is a missed dose.

2.-30. (canceled)

31. The method of claim 1, further comprising resuming a regular dosing schedule.

32. The method of claim 1, wherein the CAH is classic CAH.

33. The method of claim 1, wherein the subject is an adult.

34. The method of claim 1, wherein the subject is in a fed state.

35. The method of claim 1, wherein Compound A, or a pharmaceutically acceptable salt thereof, is administered to the subject with food.

36. The method of claim 1, wherein the first administration of Compound A, or a pharmaceutically acceptable salt thereof, is with breakfast.

37. The method of claim 1, wherein the second administration Compound A, or a pharmaceutically acceptable salt thereof, is with an evening meal.

38. The method of claim 1, wherein the first administration of Compound A, or a pharmaceutically acceptable salt thereof, is with breakfast and the second administration of Compound A, or a pharmaceutically acceptable salt thereof, is with an evening meal.

39. The method of claim 1, wherein the subject is concurrently receiving a dose of a glucocorticoid.

40. The method of claim 1, wherein the missed dose is administered as soon as possible.

41. The method of claim 1, wherein the missed dose is administered soon before the next scheduled dose.

42. The method of claim 1, wherein the subject missed administration of multiple doses.

43. A method of administering 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine, 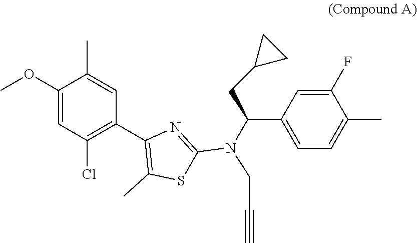
as an adjunct to glucocorticoid therapy in an adult subject having congenital adrenal hyperplasia (CAH), said method comprising: administering to the subject a first amount of Compound A for a first period of time, and subsequently administering to the subject a second amount of Compound A for a second period of time, wherein the second amount is equivalent to about 200 mg of Compound A free base q.P.M, and wherein the first amount of Compound A is a missed dose.

44. The method of claim 43, further comprising resuming a regular dosing schedule.

45. The method of claim 43, wherein the CAH is classic CAH.

46. The method of claim 43, wherein the subject is an adult.

47. The method of claim 43, wherein the subject is in a fed state.

48. The method of claim 43, wherein Compound A is administered to the subject with food.

49. The method of claim 43, wherein the first administration of Compound A is with breakfast.

50. The method of claim 43, wherein the second administration Compound A is with an evening meal.

51. The method of claim 43, wherein the first administration of Compound A is with breakfast and the second administration of Compound A is with an evening meal.

52. The method of claim 43, wherein the subject is concurrently receiving a dose of a glucocorticoid.

53. The method of claim 43, wherein the missed dose is administered as soon as possible.

54. The method of claim 43, wherein the missed dose is administered soon before the next scheduled dose.

55. The method of claim 43, wherein the subject missed administration of multiple doses.

56. A method of treating classic congenital adrenal hyperplasia in a subject in need thereof wherein said subject is administered twice daily a dose of Compound A: 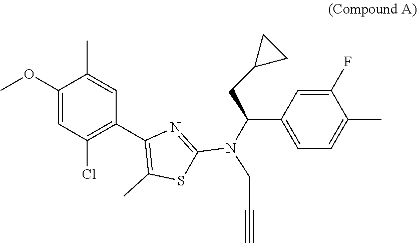
or a pharmaceutically acceptable salt thereof,
and wherein the subject has missed a dose of Compound A, comprising:
administering to the subject the amount of the missed dose of Compound A, and resuming the twice daily administration of Compound A, or a pharmaceutically acceptable salt thereof.

57. The method of claim 56, wherein the twice daily administration comprises administration of Compound A, or a pharmaceutically acceptable salt thereof, in an amount equivalent to 100 mg bid, based on the weight of the free base.

58. The method of claim 56, wherein the twice daily administration comprises administration of Compound A, or a pharmaceutically acceptable salt thereof, in an amount equivalent to 100 mg q.A.M. and an amount equivalent to 100 mg q.P.M, each based on the weight of the free base.

59. The method of claim 56, wherein the subject is an adult.

60. The method of claim 56, wherein the subject is in a fed state.

61. The method of claim 56, wherein Compound A, or a pharmaceutically acceptable salt thereof, is administered to the subject with food.

62. The method of claim 56, wherein the first administration of Compound A, or a pharmaceutically acceptable salt thereof, is with breakfast.

63. The method of claim 56, wherein the second administration Compound A, or a pharmaceutically acceptable salt thereof, is with an evening meal.

64. The method of claim 56, wherein the first administration of Compound A, or a pharmaceutically acceptable salt thereof, is with breakfast and the second administration of Compound A, or a pharmaceutically acceptable salt thereof, is with an evening meal.

65. The method of claim 56, wherein the subject is concurrently receiving a dose of a glucocorticoid.

66. The method of claim 56, wherein the missed dose is administered as soon as possible.

67. The method of claim 56, wherein the missed dose is administered soon before the next scheduled dose.

68. The method of claim 56, wherein the subject missed administration of multiple doses.

69. A method of treating classic congenital adrenal hyperplasia in a subject in need thereof wherein said subject is administered twice daily a dose of Compound A: 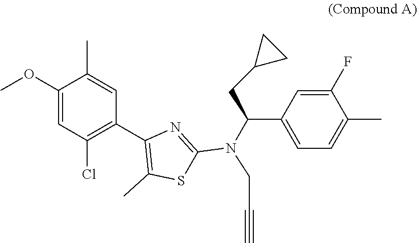
and wherein the subject has missed a dose of Compound A, comprising:
administering to the subject the amount of the missed dose of Compound A as soon as possible and
resuming the twice daily administration of Compound A.

70. The method of claim 69, wherein the twice daily administration comprises administration of Compound A in an amount equivalent to 100 mg bid.

71. The method of claim 69, wherein the twice daily administration comprises administration of Compound A in an amount equivalent to 100 mg q.A.M. and an amount equivalent to 100 mg q.P.M.

72. The method of claim 69, wherein the subject is an adult.

73. The method of claim 69, wherein the subject is in a fed state.

74. The method of claim 69, wherein Compound A is administered to the subject with food.

75. The method of claim 69, wherein the first administration of Compound A is with breakfast.

76. The method of claim 69, wherein the second administration of Compound A is with an evening meal.

77. The method of claim 69, wherein the first administration of Compound A is with breakfast and the second administration of Compound A is with an evening meal.

78. The method of claim 69, wherein the subject is concurrently receiving a dose of a glucocorticoid.

79. The method of claim 69, wherein the missed dose is administered as soon as possible.

80. The method of claim 69, wherein the missed dose is administered soon before the next scheduled dose.

81. The method of claim 69, wherein the subject missed administration of multiple doses.
